(19) United States
(12) Patent Application Publication
(10) Pub. No.: US 2026/0355391 A1
(43) Pub. Date: Nov. 12, 2026

(54) MEDICAL DEVICE WITH LUBRICIOUS LINER

(71) Applicant: Boston Scientific Scimed Inc., Maple Grove, MN (US)

(72) Inventors: Lucy Carragher, Toronto (CA); Sarang Vishnu Deodhar, Waconia, MN (US); Luca Bastone Scanga, Toronto (CA); Claudia Alonzo, Toronto (CA); Kyle Logan Lemke, Hudson, WI (US); Peter Garrett Zaviska, Mounds View, MN (US); Ashreen Ishica Chand, Mississauga (CA); Miles Montgomery, East Gwillimbury (CA); Ros Thomas Brannick, Picton (CA); Stephen O'Mahony, Dubai (AE)

(21) Appl. No.: 19/671,870

(22) Filed: May 8, 2026

Related U.S. Application Data
(60) Provisional application No. 63/802,984, filed on May 9, 2025.

(51) Int. Cl. A61L 29/14; A61L 29/08

(52) U.S. Cl. CPC A61L 29/14; A61L 29/085; A61L 2400/10; A61L 2420/02; A61L 2420/08

(57) ABSTRACT

A medical device has an outer jacket having an inner surface, and a liner adjacent to the inner surface of the outer jacket. The liner has a tie layer formed from a tie polymer, the tie layer having an inner surface and an outer surface, and a HDPE layer formed from a combination of high-density polyethylene (HDPE) and an ultra-high molecular weight siloxane. The HDPE layer is immediately adjacent to the inner surface of the tie layer. The ultra-high molecular weight siloxane is present in the HDPE layer at about 0.5 wt. % to about 10 wt. %.

### CROSS-REFERENCE TO RELATED PATENT APPLICATIONS

This application claims the benefit of priority to U.S. Provisional Patent Application No. 63/802,984, filed May 9, 2025, the entire content and disclosure of which is incorporated herein by reference.

### TECHNICAL FIELD

The present disclosure relates generally to a medical device, such as a sheath or catheter, having a lubricous liner or minor lumen, and methods of making.

### BACKGROUND

In medical procedures, a sheath is a protective tube inserted into a blood vessel to facilitate insertion and exchange of payloads, such as instruments like catheters and guidewires, without repeatedly traumatizing the vessel wall. A catheter is a hollow tube that is designed to navigate blood vessels or other body cavities. Catheters can be used to assist in a wide range of procedures, including ablation, drug treatments, measurement, mapping, and device implantation.

Lubricious coatings are used extensively in medical devices. These lubricious coatings are beneficial in reducing friction with various technologies such as stents, balloons, atherectomy and thrombectomy devices as they are pushed through the tight confines of the sheath or catheter lumen. Lubricous coatings can be in the form of liners. If the inner lumen of the liner is not of sufficient lubricity, payloads such as stents can cause the liner to collapse in an accordion-like manner as they are pushed through the lumen. Additionally, increased lubricity of the inner lumen reduces the deployment force on the payloads as they are passed through the lumen, increasing the likelihood of a successful procedure.

Previously, per- and polyfluoroalkyl substances (PFAS) may have been used as lubricious coatings due to their low coefficient of friction (lubricity), wear resistance, chemical resistance, and biocompatibility. PFASs can be defined as fluorinated substances that contain at least one fully fluorinated methyl or methylene carbon atom (without hydrogen (H), chlorine (Cl), bromine (Br), or iodine (I) attached to it). With a few noted exceptions, any chemical with at least a perfluorinated methyl group (—CF3) or a perfluorinated methylene group (—CF2-) is a PFAS. The “noted exceptions” refer to a carbon atom with a —H, —Cl, —Br, or —I atom attached to it. A global database of PFASs is available from the Organization for Economic Cooperation and Development (OECD). As PFAS materials are phased out of manufacture, a replacement is needed for lubricous liners for medical devices.

### SUMMARY

In Example 1, a medical device includes an outer jacket having an inner surface and a liner adjacent to the inner surface of the outer jacket. The liner has an inner surface and an outer surface and includes a tie layer formed from a tie polymer and a HDPE layer formed from a combination of high-density polyethylene (HDPE) and an ultra-high molecular weight siloxane. The tie layer has an inner surface and an outer surface. The HDPE layer is immediately adjacent to the inner surface of the tie layer. The ultra-high molecular weight siloxane is present in the HDPE layer at about 0.5 wt. % to about 10 wt. %.

In Example 2, the medical device of Example 1, wherein the tie polymer is selected from the group consisting of maleic anhydride grafted liner low density polyethylene polymer (MAH-g-LLDPE), anhydride-modified polyolefins, acid/acrylate-modified ethylene vinyl acetate polymer, and acid modified ethylene acrylate.

In Example 3, the medical device of Example 1 or Example 2, wherein the tie layer and the HDPE layer are co-extruded.

In Example 4, the medical device of any one of Examples 1-3, wherein the ultra-high molecular weight siloxane and the HDPE are substantially uniformly dispersed throughout the HDPE layer.

In Example 5, the medical device of any one of Examples 1-4, wherein the ultra-high molecular weight siloxane is polymethyl siloxane having a viscosity from about 10 to about 50 million mm²/s.

In Example 6, the medical device of any one of Examples 1-5, wherein the HDPE layer has a wall thickness from about 0.0005 inches to about 0.01 inches (0.013 mm to 0.25 mm).

In Example 7, the medical device of any one of Examples 1-6, wherein the tie layer has a wall thickness from about 0.0002 inches (0.0051 mm) to about 0.01 inches (0.25 mm).

In Example 8, a method of making a medical device includes forming a liner by co-extruding a tie layer and a HDPE layer and forming an outer jacket on an outer surface of the liner. The tie layer has an inner surface immediately adjacent to an outer surface of the HDPE layer, wherein the tie layer is formed from a tie polymer and the HDPE layer is formed from a combination of HDPE and an ultra-high molecular weight siloxane, and wherein the ultra-high molecular weight siloxane is present in the HDPE layer at about 0.5 wt. % to about 10 wt. %.

In Example 9, the method of Example 8, and further comprising positioning a braid between the liner and the outer jacket.

In Example 10, the method of Example 8 or Example 9, wherein the ultra-high molecular weight siloxane is present in the HDPE layer at about 1.25 wt. % to about 5 wt. %.

In Example 11, the method of any one of Examples 8-10, wherein the ultra-high molecular weight siloxane and the HDPE are substantially uniformly dispersed throughout the HDPE layer.

In Example 12, the method of any one of Examples 8-11, wherein the ultra-high molecular weight siloxane is polymethyl siloxane having a viscosity from about 10 to about 50 million mm²/s.

In Example 13, the method of any one of Examples 8-12, wherein the HDPE layer has a wall thickness from about 0.0005 inches to about 0.01 inches (0.013 mm to 0.25 mm).

In Example 14, the method of any one of Examples 8-13, wherein the tie polymer is selected from the group consisting of maleic anhydride grafted liner low density polyethylene polymer (MAH-g-LLDPE), anhydride-modified polyolefins, acid/acrylate-modified ethylene vinyl acetate polymer, and acid modified ethylene acrylate.

In Example 15, the method of any one of Examples 8-14, wherein the tie polymer is a maleic anhydride grafted liner low density polyethylene polymer (MAH-g-LLDPE), the ultra-high molecular weight siloxane is polymethyl siloxane having a viscosity from about 10 to about 50 million mm²/s, and the density of the HDPE is greater than about 0.950 g/cm³.

In Example 16, a medical device includes an outer jacket having an inner surface and a liner adjacent to the inner surface of the outer jacket. The liner has an inner surface and an outer surface and includes a tie layer formed from a tie polymer and a HDPE layer formed from a combination of high-density polyethylene (HDPE) and an ultra-high molecular weight siloxane. The tie layer has an inner surface and an outer surface. The HDPE layer is immediately adjacent to the inner surface of the tie layer. The ultra-high molecular weight siloxane is present in the HDPE layer at about 0.5 wt. % to about 10 wt. %.

In Example 17, the medical device of Example 16, wherein the tie polymer is selected from the group consisting of maleic anhydride grafted liner low density polyethylene polymer (MAH-g-LLDPE), anhydride-modified polyolefins, acid/acrylate-modified ethylene vinyl acetate polymer, and acid modified ethylene acrylate.

In Example 18, the medical device of Example 16, wherein the tie layer and the HDPE layer are co-extruded.

In Example 19, the medical device of Example 16 wherein the ultra-high molecular weight siloxane and the HDPE are substantially uniformly dispersed throughout the HDPE layer.

In Example 20, the medical device of Example 16 wherein the ultra-high molecular weight siloxane is polymethyl siloxane having a viscosity from about 10 to about 50 million mm²/s.

In Example 21, the medical device of Example 16, and further including a braid positioned between the inner surface of the outer jacket and the outer surface of the liner.

In Example 22, the medical device of Example 16, wherein ultra-high molecular weight siloxane is present in the HDPE layer at about 1.25 wt. % to about 5 wt. %.

In Example 23, the medical device of Example 16, wherein the liner further comprises a third layer, the third layer immediately adjacent to the outer surface of the tie layer and formed from the same material as the outer jacket.

In Example 24, the medical device of Example 16, wherein the HDPE layer has a wall thickness from about 0.0005 inches to about 0.01 inches (0.013 mm to 0.25 mm).

In Example 25, the medical device of Example 16, wherein the tie layer has a wall thickness from about 0.0002 inches (0.0051 mm) to about 0.01 inches (0.25 mm).

In Example 26, a method of making a medical device includes forming a liner by co-extruding a tie layer and a HDPE layer and forming an outer jacket on an outer surface of the liner. The tie layer has an inner surface immediately adjacent to an outer surface of the HDPE layer, wherein the tie layer is formed from a tie polymer and the HDPE layer is formed from a combination of HDPE and an ultra-high molecular weight siloxane, and wherein the ultra-high molecular weight siloxane is present in the HDPE layer at about 0.5 wt. % to about 10 wt. %.

In Example 27, the method of Example 26, and further comprising positioning a braid between the liner and the outer jacket.

In Example 28 the method of Example 26, wherein the ultra-high molecular weight siloxane is present in the HDPE layer at about 1.25 wt. % to about 5 wt. %.

In Example 29, the method of Example 26, wherein the ultra-high molecular weight siloxane and the HDPE are substantially uniformly dispersed throughout the HDPE layer.

In Example 30, the method of Example 26, wherein the ultra-high molecular weight siloxane is polymethyl siloxane having a viscosity from about 10 to about 50 million mm²/s.

In Example 31, the method of Example 26, wherein forming the liner includes co-extruding the tie layer, the HDPE layer and a third layer, the third layer immediately adjacent to an outer surface of the tie layer and formed from the same material as the outer jacket.

In Example 32, the method of Example 26 wherein the HDPE layer has a wall thickness from about 0.0005 inches to about 0.01 inches (0.013 mm to 0.25 mm).

In Example 33, the method of Example 26, wherein the tie layer has a wall thickness from about 0.0002 inches (0.0051 mm) to about 0.01 inches (0.25 mm).

In Example 34, the method of Example 26, wherein the tie polymer is selected from the group consisting of maleic anhydride grafted liner low density polyethylene polymer (MAH-g-LLDPE), anhydride-modified polyolefins, acid/acrylate-modified ethylene vinyl acetate polymer, and acid modified ethylene acrylate.

In Example 35, the method of Example 26, wherein the tie polymer is a maleic anhydride grafted liner low density polyethylene polymer (MAH-g-LLDPE), the ultra-high molecular weight siloxane is polymethyl siloxane having a viscosity from about 10 to about 50 million mm²/s, and the density of the HDPE is greater than about 0.950 g/cm³.

While multiple examples are disclosed, still other examples in accordance with this disclosure will become apparent to those skilled in the art from the following detailed description, which shows and describes illustrative embodiments. Accordingly, the drawings and detailed description are to be regarded as illustrative in nature and not restrictive.

### BRIEF DESCRIPTION OF THE DRAWINGS

FIG. 1 is a schematic illustration of an exemplary medical device.

FIG. 2 is a sectional perspective view of an exemplary sheath.

FIG. 3 is a cross-sectional view of an exemplary liner.

FIG. 4 is a cross-sectional view of an exemplary liner.

FIG. 5 is a cross-sectional view of an exemplary sheath.

FIG. 6 is a cross-sectional view of an exemplary sheath.

While this disclosure is amenable to various modifications and alternative forms, specific embodiments have been shown by way of example in the drawings and are described in detail below. The intention, however, is not to limit the disclosure to the particular embodiments described. On the contrary, this disclosure is intended to cover all modifications, equivalents, and alternatives falling within the scope of the disclosure as defined by the appended claims.

### DETAILED DESCRIPTION

Disclosed herein is a liner for a medical device, such as a sheath or catheter, which does not contain per- and polyfluoroalkyl substances (PFAS). That is, the coating does not contain a perfluorinated methyl group (—CF3) or a perfluorinated methylene group (—CF2-). More particularly, disclosed herein is a liner which contains a layer formed from a mixture of high-density polyethylene (HDPE) and an ultra-high molecular weight siloxane. Medical devices containing the liner and methods of making are also provided. Although described in relation to a catheter or sheath, one skilled in the art would appreciate that the liner described herein could also be used in other medical devices where appropriate.

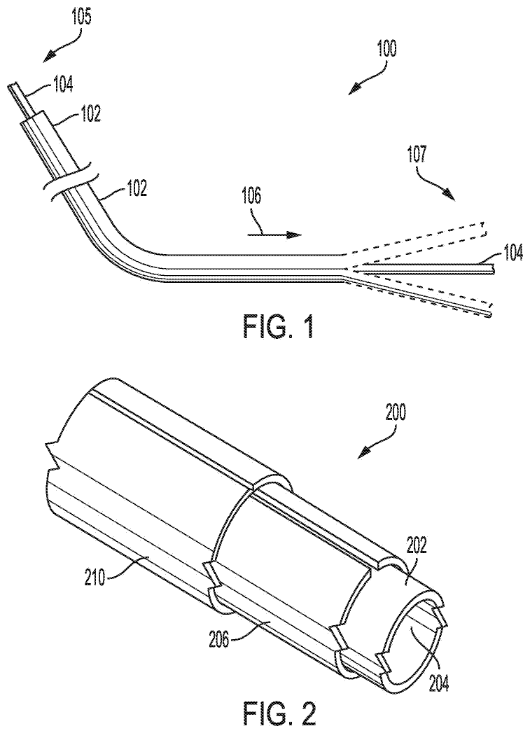

FIG. 1 is a schematic illustration of an exemplary medical device 100, such as, for example, a guide catheter. A portion of the medical device 100 in FIG. 1 is removed to show the interior. In some embodiments, the medical device 100 can be a steerable sheath or catheter, which allows for controlled maneuverability and placements of the sheath or catheter. In other embodiments, the medical device 100 can be non-steerable, such as a fixed curved sheath or a non-steerable catheter. The medical device 100 includes a sheath 102 that can be introduced into the anatomical passageways to support medical procedures. The medical procedures may include insertion of medical devices into the heart, as well as delivery of drugs, sensors, pacing leads, defibrillation leads, or other diagnostic objects into other target vessels of the body.

The sheath 102 can be a cylindrical tube, having an internal lumen for the passage of a payload 104, such as a stent, balloon, valve or other device or component. It will be appreciated that the concepts described herein may also apply to catheters and sheaths formed with a variety of cross-sectional shapes. Similarly, the concepts may be applied to shafts with additional internal features (e.g., additional lumens, steering apparatus), and shafts having features and dimensions that vary across the length of the sheath. Generally, the sheath 102 is used to place a distal end 105 of the payload 104 in an anatomical location such as a heart cavity. Once the payload 104 is implanted, the sheath 102 is withdrawn in a proximal direction, as indicated by an arrow 106, toward a proximal end 107 of the payload 104.

FIG. 2 is a sectional perspective view of a sheath 200 according to embodiments of the present disclosure. The sheath 200 includes a liner 202 that may include one or more open lumens 204 adapted for the passage of a payload. In some embodiments, the inner diameter of the liner 202 defines the lumen 204. As described herein, the liner 202 may be a multilayer liner.

In some embodiments, a braid 206 can surround the liner 202. In some embodiments, the sheath 200 can include the braid 206 embedded in the sheath wall to provide desirable physical properties to the sheath (e.g., one or more of torsional stiffness, pushing stiffness, and kink resistance). The braid 206 may be formed from round or flat metallic wires (e.g., stainless steel) or from strands of fibrous material (e.g., Vectran®, Kevlar®), for example. Metallic braids may be impregnated with a material that allows it to easily bond with polymeric materials used to form other parts of the device, such as an outer jacket as described herein. This impregnation material (e.g., Pebax® or Pebax®-nylon blends) can provide the bulk of the flexural characteristics of the produced medical device. As such, in some embodiments, the composition of the impregnation material may be varied along the length of the device to provide differing flexural characteristics to aid in guide positioning.

As shown in FIG. 2, the sheath 200 includes an outer jacket 210 surrounding the braid 206. The outer jacket 210 may form a smooth outer surface on the sheath 200.

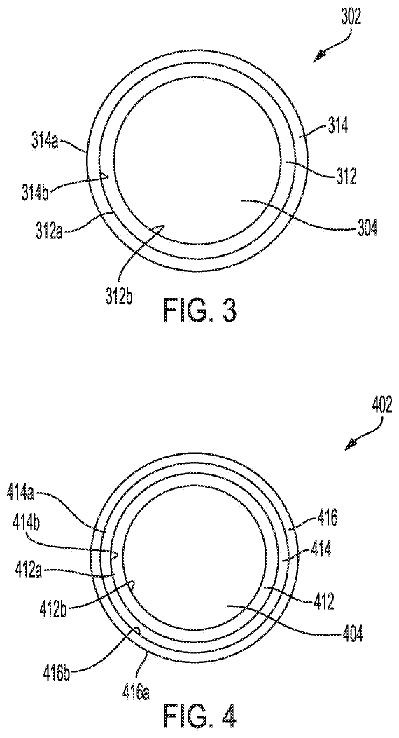

FIG. 3 is a cross-sectional view of one embodiment of the liner 202 from FIG. 2. FIG. 3 shows a liner 302 including a HDPE layer 312 (having an outer surface 312a and an inner surface 312b) and tie layer 314 (having an outer surface 314a and an inner surface 314b). A lumen 304 extends through the liner 302 and is defined by the inner surface 312b of the HDPE layer 312.

The liner 302 is a dual layer liner. In some embodiments, as described herein, the liner 302 can be formed by co-extruding the HDPE layer 312 and the tie layer 314.

The HDPE layer 312 can be a thin layer. In some embodiments, the HDPE layer 312 can have a thickness from about 0.0005 inches to about 0.01 inches (0.013 mm to 0.25 mm), about 0.0025 inches to about 0.005 (0.064 to 0.13 mm) or from about 0.001 inches to about 0.0035 inches (0.025 mm to about 0.089 mm).

The HDPE layer 312 contains a combination of high-density polyethylene (HDPE) and an ultra-high molecular weight siloxane. The geometry of HDPE causes the material to have a high tensile strength due to intermolecular forces. For example, HDPE is composed of long un-branched or slightly branched chains with little cross linkage between the molecules. As such, the polyethylene molecules of HDPE tend to lie in parallel or to form a folded molecular chain crystal structure. The density of HDPE is higher than branched lower density variants, such as LDPE. In some embodiments, the density of HDPE is greater than 0.93 g/cm³. In some embodiments, the density of HDPE is from about 0.93 g/cm³ to about 0.98 g/cm³ or from about 0.95 g/cm³ to about 0.98 g/cm³. Those skilled in the art will be able to determine the appropriate temperature and heat duration applicable to HDPE processing and their construction.

The HDPE layer 312 also contains an ultra-high molecular weight siloxane. Ultra-high molecular weight siloxane can be defined as polymethyl siloxane having an average molecular weight approaching 1 million Dalton. Ultra-high molecular weight siloxane can have a viscosity from about 10 to 50 million mm²/s (Centistokes). In some embodiments, a suitable ultra-high molecular weight siloxane is not cross linked and can exhibit flexibility similar to lower molecular weight siloxanes. In some embodiments, the ultra-high molecular weight siloxane shows greater chain entanglement and mechanical integrity than that of lower molecular weight siloxanes. Due to the ultra-high molecular weight siloxane's low surface tension and high free volume, these materials can impart the effect of lubricity more effectively than that of lower molecular weight siloxanes.

In some embodiments, the HDPE layer 312 contains from about 0.5 wt. % to about 10 wt. %, from about 1 wt. % to about 7.5 wt. %, from about 1.25 wt. % to about 5 wt. % or from about 2.5 wt. % to about 5 wt. % ultra-high molecular weight siloxane. In some embodiments, the remainder of the HDPE layer 312 is HDPE. In some embodiments, a greater amount of ultra-high molecular weight siloxane may impact the processibility of the HDPE layer 312 during manufacturing. The amount of ultra-high molecular weight siloxane also impacts the levels of D4, D5 and D6 present in HDPE layer 312 as discussed further below. Additionally, or alternatively, lower amounts of ultra-high molecular weight siloxane may result in the inner diameter of the HDPE layer 312 not having sufficient lubricity for certain materials.

Lubricity can be quantified by the coefficient of friction, which depends on the material in contact with or interacting with the HDPE layer 312. The coefficient of friction can be determined using the pinch test, as described herein. In some embodiments, the coefficient of friction of the HDPE layer 312 alone (i.e., outside the construction of the liner 302) can be from about from about 0.26 to about 0.49 when the interacting material is 7233 PEBAX®. In some embodiments, the coefficient of friction of the HDPE layer 312 alone can be from about 0.13 to about 0.26 when the interacting material is HDPE. In some embodiments, the coefficient of friction of the HDPE layer 312 alone can be from about 0.07 to about 0.16 when the interacting material is PTFE.

The ultra-high molecular weight siloxane may be substantially uniformly dispersed throughout the HDPE layer 312. As described herein, uniformity of the siloxane dispersion can be achieved in some embodiments by the process described herein. In some embodiments, uniformity of the siloxane dispersion can be determined by Thermogravimetric analysis (TGA), for example, by measuring ash content (residue left after controlled burning of the resin in TGA). Scanning electron microscopy (SEM), coupled with energy dispersive X-ray spectroscopy (EDX or EDS) can also be used to check the siloxane dispersion of the HDPE layer 312 at the micro level. In other embodiments, Fourier transform infrared spectroscopy (FTIR) analysis can be used to measure the uniformity of the siloxane dispersion. In some embodiments, the weight concentration of the siloxane does not vary by more than 10%, 5%, 3% or 1%±0.5% within HDPE layer 312.

In some embodiments, the HDPE layer 312 has acceptable levels of D4, D5 and D6, which are cyclosiloxanes mainly used as intermediate species for silicone polymer production. In some embodiments, the HDPE layer 312 contains less than or equal to 0.2 wt. % of each of D4, D5 and D6 by weight of the HDPE layer 312. In some embodiments, the HDPE layer 312 contains less than or equal to 0.05 wt. %, 0.03 wt. % or 0.01 wt. % of each of D4, D5 and D6 by weight of the HDPE layer 312. In some embodiments, the HDPE layer 312 contains less than or equal to 0.07 wt. %, 0.05 wt. % or 0.03 wt. % total D4, D5 and D6 by weight of the HDPE layer 312.

The tie layer 314 is adjacent to the HDPE layer 312. As shown in FIG. 3, the inner surface 314b of the tie layer 314 can be immediately adjacent to the outer surface 312a of the HDPE layer 312. As described herein, the tie layer 314 and the HDPE layer 312 can be coextruded such that the layers are melted or bonded at their interface.

The tie layer 314 is formed from or includes a tie polymer. In some embodiments, the tie layer 314 consists essentially of or consists of one or more tie polymers. In some embodiments, the tie layer 314 consists essentially of or consists of one tie polymer. In some examples, a suitable tie polymer is a maleic anhydride grafted liner low density polyethylene polymer (MAH-g-LLDPE), such as REZILOK RX® 101 and REZILOK RX® 102 available from Compounding Solutions, LLC of Lewiston, ME. In some embodiments, anhydride-modified polyolefins (e.g., PLEXAR® from LyondellBasell), acid/acrylate-modified ethylene vinyl acetate polymer (e.g., BYNEL™ 3101 from Dow Chemical Company), and acid modified ethylene acrylate (BYNEL™ 2002 from DOW chemical Company) can also be suitable tie polymers.

In some embodiments, the tie layer 314 can be a thin layer. In some embodiments, tie layer 314 can have a thickness from about 0.0002 inches (0.0051 mm) to about 0.01 inches (0.25 mm), from about 0.0005 inches (0.013 mm) to about 0.002 inches (0.051 mm) or from about 0.0007 inches (0.018 mm) to about 0.001 inches (0.025 mm).

One skilled in the art will recognize that the overall thickness of the liner 302 and the individual thicknesses of the HDPE layer 312 and the tie layer 314 can be varied to provide desired characteristics for the medical device. In some embodiments, the medical device may be less stable if the thickness of the liner 302 is too thin. On the other hand, the medical device may become undesirably stiff if the thickness of liner 302 is too great.

In some embodiments, the inclusion of the ultra-high molecular weight siloxane can increase the lubricity of the HDPE layer 312. For example, the inclusion of the ultra-high molecular weight siloxane can reduce the average coefficient of friction of HDPE layer 312 alone compared to a layer formed of the HDPE material without the ultra-high molecular weight siloxane when the interacting material is HDPE. In some embodiments, the lubricity of the liner 302, as measured through the lumen 304, is sufficient for medical use. In some embodiments, the liner 302 has a lubricity similar to the lubricity of a liner formed from PTFE. In some embodiments, the lubricity of the liner 302 can be evaluated by peak track force where a lower peak track force indicates a greater lubricity. In some embodiments, liner 302 has a peak track force or an average peak track force within +17%, 15%, 10%, or 5% of the peak track force or average peak track force of a PTFE liner using a rigid model.

In some embodiments, the liner 302 has a durability similar to the durability of liner formed from PTFE. One way to test durability of the liner 302 is to repeat the lubricity test many times or cycles. A liner in which the lubricity reduces more quickly over the cycles has a worse durability.

In some embodiments, the liner 302 does not impair the functionality of the medical device as compared to a liner currently used in the device. For example, a sheath containing the liner 302 may maintain sufficient flexibility and durability for medical use. In some embodiments, the sufficiency can be determined by comparing the use of the liner 302 with the currently used liner, such as a PTFE liner. Thus, in some embodiments, the liner 302 may be sufficient to replace current PTFE liners in medical devices.

In some embodiments, the tie layer 314 can assist in bonding the HDPE layer 312 to another component of the medical device, such as an outer jacket or a third layer of the liner as described herein. In some embodiments, the HDPE layer 312 and the other component may be formed of different and/or dissimilar polymers. The tie polymer of the tie layer 314 may be selected to improve bonding of the HDPE layer 312 to the other component as described herein.

FIG. 4 is a cross-sectional view of another embodiment of liner 202 from FIG. 2. FIG. 4 is a cross-sectional view of a liner 402 having a tri-layer design. The liner 402 includes a HDPE layer 412 (having an outer surface 412a and an inner surface 412b), a tie layer 414 (having an outer surface 414a and an inner surface 414b) and a third layer 416 (having an outer surface 416a and an inner surface 416b). A lumen 404 is defined by the inner surface 412b of the HDPE layer 412.

The HDPE layer 412 and the tie layer 414 are similar to the HDPE layer 312 and the tie layer 314 described with respect to FIG. 3. The third layer 416 is adjacent to the tie layer 414. As shown in FIG. 4, the outer surface 414a of the tie layer 414 can be immediately adjacent to the inner surface 416b of the third layer 416. The HDPE layer 412, the tie layer 414 and the third layer 416 can be coextruded such that the layers are melted or bonded at their respective interfaces.

The third layer 416 can be formed of the same material as the outer jacket 210. Suitable materials include polyamide thermoplastic elastomers (TPE) such as VESTAMID® ML resin from Evonik, polyether block amides (PEBA) TPE such as PEBAX® grades from Arkema and VESTAMID® ME grades from Evonik, TPE polyurethanes (TPE PU) such as PELLATHANE® and TECOFLEX™ from Lubrizol, and co-polyester TPE such as HYTREL® from DuPont and ARNITEL® from DSM. In some embodiments, the third layer 416 can be formed from a material capable of withstanding higher temperatures than other layers of the liner 402. In this way, the third layer 416 can provide thermal stability to the liner 402.

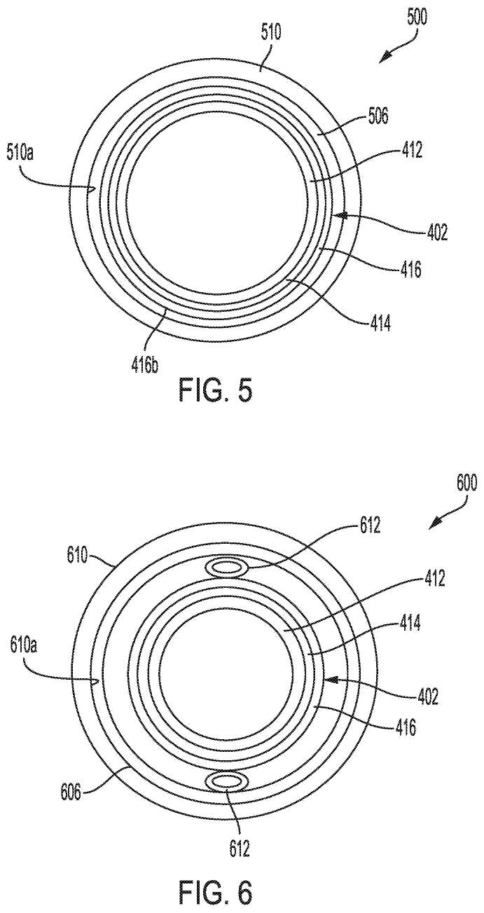

FIG. 5 is a cross-sectional view of an embodiment of a sheath 500 which includes the liner 402, a braid 506 and an outer jacket 510. While the liner 402, which has a tri-layer design, is shown, one skilled in the art will recognize that the liner 302, which has a dual-layer design, could also be used in the sheath 500.

As discussed herein, the liner 402 includes the HDPE layer 412, the tie layer 414 and the third layer 416. The third layer 416 is adjacent to the braid 506. In some embodiments, the third layer 416 is immediately adjacent to the braid 506, and the braid 506 is in contact with the outer surface 416a of the third layer 416. In some embodiments, the outer jacket 510 is immediately adjacent to the braid 506, and the braid 506 is in contact with an inner surface 510a of the outer jacket 510.

In some embodiments, liner 402 sufficiently bonds to the remaining assembly. For example, the liner 402 sufficiently bonds to the braid 506 and the outer jacket 510. In some embodiments, the tie layer 414 of the liner 402 assists in bonding the HDPE layer 412 to the outer jacket 510 and/or the third layer 416 of the liner 402. In some embodiments, the HDPE layer 412 and the outer jacket 510 may be formed of different and/or dissimilar polymers. The tie polymer of the tie layer 414 may be selected to improve bonding of the HDPE layer 412 to the outer jacket 510.

In some embodiments, the sheath 500 can be formed by forming the liner 402 followed by formation of the remainder of the device, such as positioning of the braid 506 and formation of the outer jacket 510.

In some embodiments, the liner 402 can be formed by coextruding layers of the liner 402. Where the liner 402 is a tri-layer design, the HDPE layer 412, the tie layer 414 and the third layer 416 can be coextruded through a die or using other suitable equipment. In some embodiments, the HDPE layer 412, the tie layer 414 and the third layer 416 are coextruded at a temperature above the melt temperature of each respective layer. In this way, the layers are melted or bonded together in the liner 402. In some embodiments, the die size determines or affects the draw, draw ratio balance (DRB) and draw down ratio (DDR) which in turn affect the process stability and obtaining parts within dimensional specifications. One skilled in the art will recognize that various die sizes can be used depending on the desired liner size. In some embodiments, the die size can range from about 0.015 inches to about 0.02 inches (0.381 mm to 0.508 mm). One skilled in the art will also recognize that the liner 302 can be formed in substantially the same process but without the third layer 416.

In some embodiments, the HDPE layer 412 can be formed by extruding a compounded material containing HDPE and a masterbatch material containing ultra-high molecular weight siloxane. In some embodiments, the ultra-high molecular weight (UHMW) siloxane can be compounded with one or more polymers, such as polypropylene, HDPE, LDPE, Nylon 6, co-Polyester, to create the masterbatch. The masterbatch is blended with HDPE and coextruded with the remaining layers of the liner 402. Selection of the masterbatch depends upon type of bulk polymer. For example, in some embodiments, masterbatch MULTIBASE™ MB50-314 and MULTIBASE™ MB25-235, available from Dupont, are suitable to blend a HDPE polymer.

In some embodiments, the liner 402 can be formed by continuous coextrusion. For example, the HDPE layer 412, the tie layer 414 and the third layer 416 can be coextruded and spooled. When the liner 402 is formed by continuous extrusion, the length of appropriate liner can be cut from the spool before proceeding to the next step in making the sheath. In other embodiments, the liner 402 can be formed by discrete coextrusion. In discrete manufacturing, the HDPE layer 412, the tie layer 414 and the third layer 416 can be coextruded and cut to length at time of extrusion.

After liner 402 is formed, the braid 506 is positioned on the liner 402. After the braid 506 is positioned, the outer jacket 510 can be formed on the braid 506. In some embodiments, the material of the outer jacket 510 can penetrate through the openings in the braid 506 and attach or bond to the third layer 416. In some embodiments, the outer jacket 510 can be formed by extrusion. In some embodiments the outer jacket 510 can go through a secondary process to smooth the outer surface. For example, a heated die which has a slightly smaller inner diameter than the outer diameter of the outer jacket 510 can be used to smooth the outer surface. This process can remove any texture on the outer surface of the outer jacket 510 and ensure a smooth outer surface.

FIG. 6 is a cross-sectional view of a sheath 600 which includes minor lumens or pull-wire lumens 612 positioned between the liner 402 and a braid 606. One skilled in the art will recognize that FIG. 6 is prior to reflow or bonding of the outer jacket. In some embodiments, the third layer 416 can provide thermal integrity to the assembly for example by preventing the minor lumens 612 from being consumed by or melting into the liner 402 upon reflow. For example, the assembly can be exposed to elevated temperatures during the reflow process and the higher melt temperature of the third layer 416 as compared to other layers of the liner 402 can prevent the minor lumens 612 from melting into the liner 402.

One skilled in the art will recognize that while the liner 402 has been described for the main lumen of the sheath 600, similar configurations may be used as liners for other lumens, such as minor lumens or pull-wire lumens 612.

### EXAMPLES

### Example 1—Track Data

Trackability testing measures the force necessary to move a component along the length of a simulated anatomy fixture or model. In Example 1, trackability testing was conducted on a rigid model having curves or bends in its construction to simulate the human cardiovascular anatomy from the femoral vein to the heart. Track force data can be used comparatively. In such a situation, less force indicates more trackability.

In the Example 1, an assembled sheath containing a liner with a HDPE layer was compared to sheaths having a PTFE liner which are currently used in medical devices to determine the feasibility of the HDPE layer. The liner material indicated in Table 1 is the material on the inner diameter of the sheath and exposed to the catheter. The HDPE liner in Table 1 was formed by coextruding a third layer, a tie layer and HDPE layer containing PETROTHENE® LR34002 available from Lynodell Bassel and MULTIBASE™ MB-50-314, an ultra-high molecular weight functionalized siloxane polymer dispersed in high density polyethylene, available from Dupont at a temperature above the melt temperature of the HDPE polymer. A braid and outer jacket were formed on the sheath as described herein.

To measure the track force, the assembled sheath was positioned in the model and force required to push a representative catheter through the sheath was measured. The same representative catheter was used for Test Conditions 1-3 and for Test Conditions 4-8 to allow comparison of the data within the sample sets. The sample size indicates the number of samples or devices tested for each test condition; peak track forces were measured in a minimum of three runs per sample. A range of the peak track force measured is provided in Table 1. The average peak track force is also provided.

For Tests Conditions 1 and 2 (referred to as Test 1 and Test 2), a 13 French (Fr) (4.33 mm) discrete reflow steerable sheath (inner diameter of 13 Fr; liner formed by discrete reflow process) was tested with a PTFE liner (Test 2) and a liner having an inner layer of HDPE containing 5 wt. % siloxane (a HDPE layer) (“HDPE Liner with 5% siloxane”) (Test 1). With reference to Table 1, the track forces of Test 1 (HDPE liner with 5% siloxane) were comparable to that of Test 2. This suggests that a sheath similar to sheath 500 having the HDPE layer 412 with 5 wt. % siloxane will perform similarly to sheaths that are currently in commercial use.

For Tests Conditions 3 and 4 (referred to as Test 3 and Test 4), a 8.5 Fr (2.67 mm) discrete reflow fixed sheath was tested with a representative catheter. With reference to Table 1, the track forces of Test 3 (HDPE liner with 5 wt. % siloxane) were comparable to Test 4 (PTFE liner), suggesting that the HDPE liner with 5 wt. % siloxane will perform similarly to liners that are currently in commercial use.

For Test Conditions 5-7 (referred to as Test 5, 6 and 7, respectively), a 8.5 Fr (2.67 mm) a sheath was tested with a PTFE liner (Test 5), a liner having an inner layer of HDPE and 5 wt. % siloxane (a HDPE layer) (“HDPE Liner with 5% siloxane”) (Test 6), and a liner having an inner layer of HDPE and 2.5 wt. % siloxane (a HDPE layer) (“HDPE Liner with 2.5% siloxane”) (Test 7). Test 5 was a fixed sheath and Tests 6 and 7 were continuous sheath constructions. The track forces of Tests 6-8 were comparable suggesting that the HDPE liner with 5 wt. % siloxane and 2.5 wt. % siloxane will perform similarly to liners that are currently in commercial use.

TABLE 1
Example 1 Peak Track Forces Data

| | | Peak | Average Peak | | |
|---|---|---|---|---|---|
| Test | | Track Force | Track Force | Sample | |
| Condition | Description | (grams force) | (grams force) | size | Additional details |
| 1 | HDPE Liner with | Range: 385-664 | 515 | 2 | 13Fr discrete |
| | 5% siloxane | | | | reflow steerable |
| | | | | | construction |
| 2 | PTFE Liner | Range: 443-626 | 511 | 1 | 13Fr discrete |
| | (control) | | | | reflow steerable |
| | | | | | construction |
| 3 | HDPE Liner with | Range: 145-229 | 195 | 6 | 8.5Fr discrete |
| | 5% siloxane | | | | reflowed fixed |
| | | | | | sheath |
| | | | | | construction |
| 4 | PTFE Liner | Range: 115-225 | 173 | 5 | 8.5Fr discrete |
| | (control) | | | | reflowed fixed |
| | | | | | sheath |
| | | | | | construction |
| 5 | PTFE (control) | Range: 125-228 | 144 | 2 | 8.5Fr discrete |
| | | | | | reflowed fixed |
| | | | | | sheath |
| | | | | | construction |
| 6 | HDPE Liner with | Range: 142-191 | 166 | 2 | 8.5Fr continuous |
| | 5% siloxane | | | | fixed sheath |
| | | | | | construction |
| 7 | HDPE Liner with | Range: 117-238 | 171 | 2 | 8.5Fr continuous |
| | 2.5% siloxane | | | | fixed sheath |
| | | | | | construction |

The results from Test Conditions 1-7 suggest that the HDPE layer containing liner is feasible for fixed and for steerable sheaths. The data also suggests that the HDPE layer containing liner can be constructed by a continuous or discrete method. The data further suggests that the HDPE layer containing liner is comparable to liners currently used in sheaths.

### Example 2—Coefficient of Friction

The average coefficient of friction was measured for liner materials currently used in medical devices and an HDPE layer of the liner as described herein. A Harland Medical FTS 6000 friction tester was used to measure the frictional force between a liner material and an interacting material. A liner of the composition described in Table 2 was positioned on a silicone face of a gripper pad of the tester such that the liner material covered the entire face, and no silicone was exposed. The sample was tested using a clamp force of 300 g, an elevator velocity of 1.0 cm/sec, and an elevator move distance of 8 cm. The sample was pulled through gripper pads of the specified interacting material 15 times and the average coefficient of friction across the 15 pulls was calculated.

TABLE 2
Example 2 Average Co-efficient of Friction Data

| Test | | Interacting | Average Co-efficient | Sample |
|---|---|---|---|---|
| Condition | Description | Material | of Friction | size |
| 9 | PTFE (control) | 7233 PEBAX ® | Range: 0.108-0.305 | 12 |
| 10 | HDPE Liner | 7233 PEBAX ® | Range: 0.276-0.297 | 5 |
| 11 | HDPE Liner with | 7233 PEBAX ® | Range: 0.334-0.483 | 15 |
| | 5 wt. % siloxane | | | |
| 12 | HDPE Liner with | 7233 PEBAX ® | Range: 0.304-0.453 | 15 |
| | 7.5 wt. % siloxane | | | |
| 13 | PTFE (control) | HDPE | Range: 0.087-0.096 | 3 |
| 14 | HDPE Liner | HDPE | Range: 0.219-0.251 | 5 |
| 15 | HDPE Liner with | HDPE | Range: 0.153-0.202 | 10 |
| | 5 wt. % siloxane | | | |
| 16 | HDPE Liner with | HDPE | Range: 0.142-0.239 | 15 |
| | 7.5 wt. % siloxane | | | |
| 17 | PTFE (control) | PTFE | Range: 0.134-0.146 | 12 |
| 18 | HDPE Liner | PTFE | Range: 0.085-0.113 | 5 |
| 19 | HDPE Liner with | PTFE | Range: 0.082-0.117 | 10 |
| | 5 wt. % siloxane | | | |
| 20 | HDPE Liner with | PTFE | Range: 0.129-0.154 | 10 |
| | 7.5 wt. % siloxane | | | |

The data suggests that the HDPE liner with siloxane has a co-efficient of friction similar to that of currently used liner materials for the three interacting materials tested, PEBAX®, HDPE and PTFE. The materials testing in Table 2 were the raw liner materials without the presence of additional liner layers and/or other sheath materials. For example, the HDPE Liner with 5 wt. % siloxane was a layer formed of HDPE mixed with 5 wt. % ultra-high molecular weight siloxane.

Various modifications and additions can be made to the embodiments discussed without departing from the scope of this disclosure. For example, while the embodiments described above refer to particular features, the scope of this disclosure also includes embodiments having different combinations of features and embodiments that do not include all of the described features. Accordingly, the scope of this disclosure is intended to embrace all such alternatives, modifications, and variations as fall within the scope of the claims, together with all equivalents thereof.

What is claimed is:

1. A medical device comprising:
an outer jacket having an inner surface; and
a liner adjacent to the inner surface of the outer jacket, the liner having an inner surface and an outer surface and comprising:
a tie layer formed from a tie polymer, the tie layer having an inner surface and an outer surface; and
a HDPE layer formed from a combination of high-density polyethylene (HDPE) and an ultra-high molecular weight siloxane, the HDPE layer immediately adjacent to the inner surface of the tie layer and wherein the ultra-high molecular weight siloxane is present in the HDPE layer at about 0.5 wt. % to about 10 wt. %.

2. The medical device of claim 1, wherein the tie polymer is selected from the group consisting of maleic anhydride grafted liner low density polyethylene polymer (MAH-g-LLDPE), anhydride-modified polyolefins, acid/acrylate-modified ethylene vinyl acetate polymer, and acid modified ethylene acrylate.

3. The medical device of claim 1, wherein the tie layer and the HDPE layer are co-extruded.

4. The medical device of claim 1, wherein the ultra-high molecular weight siloxane and the HDPE are substantially uniformly dispersed throughout the HDPE layer.

5. The medical device of claim 1, wherein the ultra-high molecular weight siloxane is polymethyl siloxane having a viscosity from about 10 to about 50 million mm²/s.

6. The medical device of claim 1, and further comprising a braid positioned between the inner surface of the outer jacket and the outer surface of the liner.

7. The medical device of claim 1, wherein ultra-high molecular weight siloxane is present in the HDPE layer at about 1.25 wt. % to about 5 wt. %.

8. The medical device of claim 1, wherein the liner further comprises a third layer, the third layer immediately adjacent to the outer surface of the tie layer and formed from the same material as the outer jacket.

9. The medical device of claim 1, wherein the HDPE layer has a wall thickness from about 0.0005 inches to about 0.01 inches (0.013 mm to 0.25 mm).

10. The medical device of claim 1, wherein the tie layer has a wall thickness from about 0.0002 inches (0.0051 mm) to about 0.01 inches (0.25 mm).

11. A method of making a medical device, the method comprising:
forming a liner by co-extruding a tie layer and a HDPE layer, the tie layer having an inner surface immediately adjacent to an outer surface of the HDPE layer, wherein the tie layer is formed from a tie polymer and the HDPE layer is formed from a combination of HDPE and an ultra-high molecular weight siloxane, and wherein the ultra-high molecular weight siloxane is present in the HDPE layer at about 0.5 wt. % to about 10 wt. %; and
forming an outer jacket on an outer surface of the liner.

12. The method of claim 11, and further comprising positioning a braid between the liner and the outer jacket.

13. The method of claim 11, wherein the ultra-high molecular weight siloxane is present in the HDPE layer at about 1.25 wt. % to about 5 wt. %.

14. The method of claim 11, wherein the ultra-high molecular weight siloxane and the HDPE are substantially uniformly dispersed throughout the HDPE layer.

15. The method of claim 11, wherein the ultra-high molecular weight siloxane is polymethyl siloxane having a viscosity from about 10 to about 50 million mm²/s.

16. The method of claim 11, wherein forming the liner includes co-extruding the tie layer, the HDPE layer and a third layer, the third layer immediately adjacent to an outer surface of the tie layer and formed from the same material as the outer jacket.

17. The method of claim 11, wherein the HDPE layer has a wall thickness from about 0.0005 inches to about 0.01 inches (0.013 mm to 0.25 mm).

18. The method of claim 11, wherein the tie layer has a wall thickness from about 0.0002 inches (0.0051 mm) to about 0.01 inches (0.25 mm).

19. The method of claim 11, wherein the tie polymer is selected from the group consisting of maleic anhydride grafted liner low density polyethylene polymer (MAH-g-LLDPE), anhydride-modified polyolefins, acid/acrylate-modified ethylene vinyl acetate polymer, and acid modified ethylene acrylate.

20. The method of claim 11, wherein the tie polymer is a maleic anhydride grafted liner low density polyethylene polymer (MAH-g-LLDPE), the ultra-high molecular weight siloxane is polymethyl siloxane having a viscosity from about 10 to about 50 million mm²/s, and the density of the HDPE is greater than about 0.950 g/cm³.
